(19) United States
(12) Patent Application Publication
(10) Pub. No.: US 2015/0329754 A1
(43) Pub. Date: Nov. 19, 2015

(54) LIQUID COMPOSITION

(71) Applicant: Daicel Corporation, Osaka-shi, Osaka (JP)

(72) Inventors: Masaaki ITO, Himeji-shi (JP); Ryota IWATA, Himeji-shi (JP)

(73) Assignee: DAICEL CORPORATION, Osaka-shi, Osaka (JP)

(21) Appl. No.: 14/435,962

(22) PCT Filed: Oct. 4, 2013
(86) PCT No.: PCT/JP2013/077070

§ 371 Date: Apr. 15, 2015

(30) Foreign Application Priority Data
Oct. 16, 2012 (JP) 2012-229171

(51) Int. Cl. C09K 3/00; C07C 231/14; C07C 231/02

(52) U.S. Cl. CPC C09K 3/00; C07C 231/02; C07C 231/14

(57) ABSTRACT

Provided is a liquid composition that places a low burden on the environment and has such properties as to thicken or gelate upon the application of strain. The liquid composition according to the present invention contains components (A), (B), (C), and (D). The component (A) is an N-methyl-N-(2,3-dihydroxypropyl) fatty amide represented by Formula (1). The component (B) is an N-methyl-N-(2-hydroxypropyl) fatty amide ester represented by Formula (2). The component (C) is an oily substance. The component (D) is water. Formulae (1) and (2) are expressed as follows:

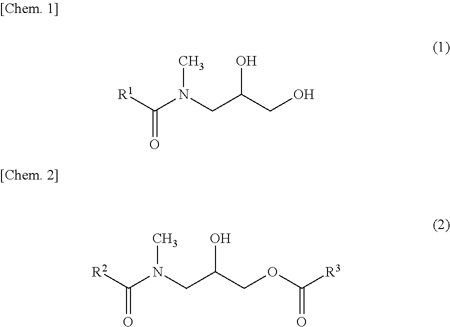

### TECHNICAL FIELD

The present invention relates to liquid compositions having shear-thickening properties.

### BACKGROUND ART

Of materials having nonlinear rheological properties, liquid compositions including a branched polymeric compound and/or a water-soluble polymeric compound generally have non-Newtonian properties and are widely applied to coating materials and coating agents using pseudoplasticity; and a variety of emulsified compositions using thixotrophy, such as toothpaste, inks, and cosmetic creams. These compositions show shear-thinning properties as to have a reducing viscosity upon the application of strain at a certain level or more. Disadvantageously, such compositions having shear-thinning properties scatter upon traveling at a high speed on a production line so as to offer better productivity.

The nonlinear rheological properties include the shear-thinning properties; and shear-thickening properties to offer a higher viscosity or to become galated upon the application of strain at a certain level or more. Liquid compositions having shear-thickening properties are useful typically in areas of dental materials, coating compositions, sealing materials, viscoelastic abrasive media, materials for devices with silencing function (noise-reducing function), filling materials for display deformation restraining layers, printer inks, cosmetic base materials, and impact absorbing materials (Patent Literature (PTL) 1 to 12 and Non Patent Literature (NPL) 1).

However, there are known few liquid compositions having shear-thickening properties. This impedes the provision of sufficient options for various technical requirements in the areas under present circumstances. Already-known liquid compositions having shear-thickening properties develop a shear-thickening property such as rheopexy or dilatancy by the interaction between inorganic fine particles and a polysiloxane, or by the interaction between microemulsion particles and a water-soluble polymeric compound, or by the interaction between ionic polymeric compounds. However, the use of the components such as the inorganic fine particles, polysiloxane, and ionic polymeric compound is not desirable in some cases.

The liquid compositions having shear-thickening properties are considered to develop shear-thickening properties in a manner as follows. A polymeric compound has an entropically stable, coiled structure under no strain, but is linearly elongated upon the application of strain to a solution containing the polymeric compound. This triggers and causes the interaction with fine particles, emulsion, or another polymer (polymeric compound) to form crosslinking points to thereby develop the shear-thickening properties. In general, such polymeric compound, when applied with strain stress, undergoes molecular chain cleavage. Disadvantageously, this causes the polymeric compound to develop shear-thickening properties with inferior power after repeated use. Further disadvantageously, the interaction between such polymeric compound with a microemulsion formed using an ionic surfactant, and the interaction between ionic polymeric compounds have properties that vary depending on the species and concentration of an ion coexisting in a use environment or in the product composition. It is also pointed out that the polymeric compounds contained in the liquid compositions having shear-thickening properties are resistant to biodegradation and may possibly cause environmental pollution.

### CITATION LIST

### Patent Literature

PTL 1: Japanese Unexamined Patent Application Publication (JP-A) (Translation of PCT Application) No. 2001-509179
PTL 2: JP-A No. 2010-082563
PTL 3: JP-A No. 2010-259811
PTL 4: JP-A (Translation of PCT Application) No. 2010-516900
PTL 5: JP-A No. 2009-298366
PTL 6: JP-A No. 2009-108940
PTL 7: JP-A No. 2009-243324
PTL 8: JP-A No. 2009-230072
PTL 9: JP-A No. 2008-138018
PTL 10: JP-A No. 2008-214390
PTL 11: JP-A No. 2010-144021
PTL 12: JP-A No. 2010-18660

### Non Patent Literature

NPL 1: FRAGRANCE JOURNAL, vol. 40 (No. 3) pp. 43-49 (2012)

### SUMMARY OF INVENTION

### Technical Problem

Accordingly, it is an object of the present invention to provide a liquid composition that places a low burden on the environment and has such properties as to thicken (to have a higher viscosity) or gelate upon application of strain.

It is another object of the present invention to provide a method for producing an N-methyl-N-(2,3-dihydroxypropyl) fatty amide and an N-methyl-N-(2-hydroxypropyl) fatty amide ester, both acting as components for the liquid composition.

### Solution to Problem

After intensive investigations to achieve the objects, the present inventors have found a liquid composition that contains an N-methyl-N-(2,3-dihydroxypropyl) fatty amide, an N-methyl-N-(2-hydroxypropyl) fatty amide ester, an oily substance, and water, where the N-methyl-N-(2,3-dihydroxypropyl) fatty amide contains one hydrophobic group and one hydrophilic groups per molecule, and the N-methyl-N-(2-hydroxypropyl) fatty amide ester contains two hydrophobic groups and one hydrophilic group per molecule. The present inventors have found as follows. This liquid composition offers such shear-thickening properties as to be liquid in a stationary state, but to thicken or gelate upon the application of strain even when the liquid composition is devoid of polymeric compounds placing high environmental burdens. The properties are developed because the applied strain induces the change of higher-order aggregation structure of the low-molecular organic compounds. Accordingly, the liquid composition resists the fracture of its primary molecular structure and does not undergo deterioration in developing power of the the properties even after repeated use. The present inventors have also found as follows. Assume that an amine and a fatty acid ester are allowed to react with each other in specific proportions in the presence of a basic catalyst. This enables one-pot production of a mixture of the N-methyl-N-(2,3-dihydroxypropyl) fatty amide and the N-methyl-N-(2-hydroxypropyl) fatty amide ester. The present invention has been made based on these findings.

Specifically, the present invention provides, in one aspect, a liquid composition containing components (A), (B), (C), and (D). The component (A) is an N-methyl-N-(2,3-dihydroxypropyl) fatty amide represented by Formula (1):

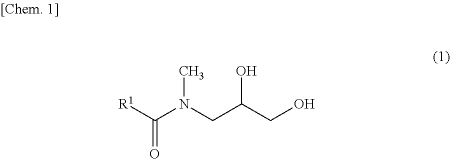

where R¹ represents C₅-C₂₀ straight or branched chain alkyl or alkenyl. The component (B) is an N-methyl-N-(2-hydroxypropyl) fatty amide ester represented by Formula (2):

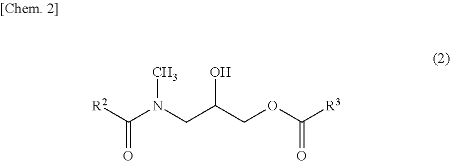

where R² and R³ independently represent, identically or differently, C₅-C₂₀ straight or branched chain alkyl or alkenyl. The component (C) is an oily substance. The component (D) is water.

The ratio (weight ratio) of the component (A) to the component (B) is preferably from 99:1 to 70:30.

The component (C) preferably includes a straight or branched chain hydrocarbon oil.

The liquid composition according to the present invention may have a first storage modulus [G′ (Pa)] at a shear rate of 350 rad/s, a second storage modulus [G′ (Pa)] at a shear rate of 1 rad/s, a first loss modulus [G″ (Pa)] at a shear rate of 350 rad/s, and a second loss modulus [G″ (Pa)] at a shear rate of 1 rad/s as determined at a temperature of from 10° C. to 50° C. In this case, the difference between the first and second storage moduli is preferably 20 Pa or more, and the difference between the first and second loss moduli is preferably 10 Pa or more.

The liquid composition according to the present invention preferably further contains an additional component (E). The component (E) may include an amphiphilic substance excluding compounds belonging to the components (A) and (B).

The present invention provides, in another aspect, a method for producing a fatty amide and a fatty amide ester. The method includes allowing an amine represented by Formula (3) to react with a fatty acid ester represented by Formula (4a) in an amount of from 0.95 mole to less than 1.95 moles per mole of the amine in the presence of a basic catalyst. This gives both an N-methyl-N-(2,3-dihydroxypropyl) fatty amide represented by Formula (1a) and an N-methyl-N-(2-hydroxypropyl) fatty amide ester represented by Formula (2a). Formulae (3), (4), (1a), and (2a) are expressed as follows:

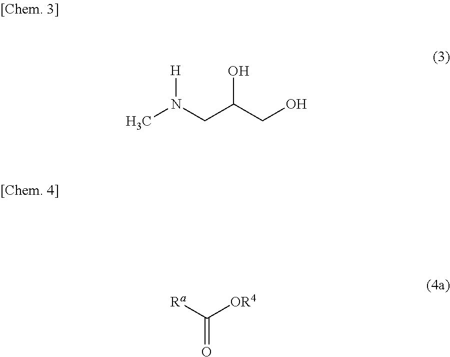

where R^{a} represents C₅-C₂₀ straight or branched chain alkyl or alkenyl; and R⁴ represents a hydrocarbon group,

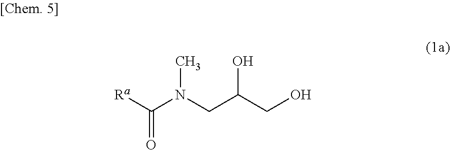

where R^{a} is as defined above,

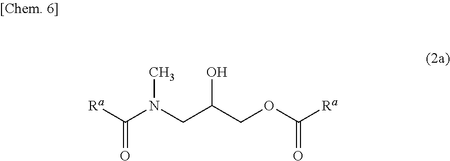

where R^{a} is as defined above.

The present invention provides, in yet another aspect, a method for producing a fatty amide. The method includes allowing an amine represented by Formula (3) to react with a fatty acid ester represented by Formula (4b) in an amount of 1.2 moles or less per mole of the amine in the presence of a basic catalyst. This gives an N-methyl-N-(2,3-dihydroxypropyl) fatty amide represented by Formula (1). Formulae (3), (4b), and (1) are expressed as follows:

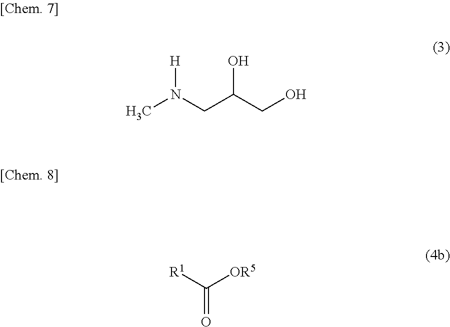

where R¹ represents C₅-C₂₀ straight or branched chain alkyl or alkenyl; and R⁵ represents a hydrocarbon group,

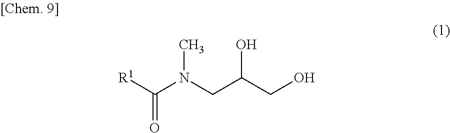

where R¹ is as defined above.

In addition and advantageously, the present invention provides a method for producing a liquid composition. The method includes preparing a mixture of the N-methyl-N-(2,3-dihydroxypropyl) fatty amide represented by Formula (1a) and the N-methyl-N-(2-hydroxypropyl) fatty amide ester represented by Formula (2a) by the method for producing a fatty amide and a fatty amide ester. The resulting mixture is mixed with an oily substance and water. Formulae (1a) and (2a) are expressed as follows:

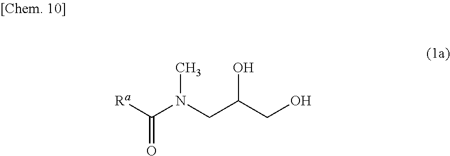

where R^{a} represents C₅-C₂₀ straight or branched chain alkyl or alkenyl,

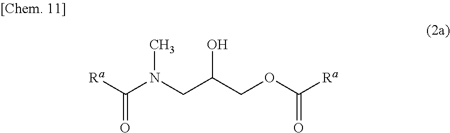

where R^{a} is as defined above.

### Advantageous Effects of Invention

The liquid composition according to the present invention, as having the configuration, characteristically develops shear-thickening properties with good sensitivity and can maintain the properties over a long period of time. The liquid composition does not require the use of polymeric compounds placing high burdens on the environment and is environmental-friendly. The liquid composition according to the present invention is advantageously usable as rheology control materials in or for materials requiring shear-thickening properties. Such materials are exemplified by impact absorbing material fillers, soling materials, viscoelastic abrasive media, materials for devices with silencing function (noise-reducing function), filling materials for display deformation restraining layers, printer inks, coating compositions, cosmetic base materials, and dental materials.

### BRIEF DESCRIPTION OF DRAWINGS

FIG. 1 is a graph illustrating how the storage modulus (G′) varies depending on the shear rate (ω) at temperatures of 10° C., 30° C., and 50° C. in a composition prepared in Example 8; and

FIG. 2 is a graph illustrating how the loss modulus (G″) varies depending on the shear rate (ω) at temperatures of 10° C., 30° C., and 50° C. in the composition prepared in Example 8.

### DESCRIPTION OF EMBODIMENTS

The liquid composition according to the present invention contains components (A), (B), (C), and (D) as follows. The component (A) is an N-methyl-N-(2,3-dihydroxypropyl) fatty amide represented by Formula (1). The component (B) is an N-methyl-N-(2-hydroxypropyl) fatty amide ester represented by Formula (2). The component (C) is an oily substance. The component (D) is water.

Component (A)

The component (A) for use in the present invention is the N-methyl-N-(2,3-dihydroxypropyl) fatty amide represented by Formula (1).

In Formula (1), R¹ represents C₅-C₂₀ straight or branched chain alkyl or alkenyl. The R¹C(═O) group is exemplified by n-pentanoyl, isopentanoyl, n-hexanoyl, n-octanoyl, isooctanoyl, 2-ethylhexanoyl, 3-ethylhexanoyl, n-nonanoyl, isononyl, n-decanoyl, isodecanoyl, n-undecanoyl, n-dodecanoyl, n-tetradecanoyl, n-hexadecanoyl, n-icosanoyl, n-pentenoyl, isopentenoyl, n-hexenoyl, n-octenoyl, isooctenoyl, 2-ethylhexenoyl, 3-ethylhexenoyl, n-nonenoyl, isononenoyl, n-decenoyl, isodecenoyl, n-undecenoyl, n-dodecenoyl, n-tetradecenoyl, n-hexadecenoyl, and n-icosenoyl groups.

Among them, R¹ in Formula (1) is preferably C₉-C₁₅ straight chain alkyl for excellent fluidity and temperature stability (thermal stability). The group as R¹, if containing carbon atoms in a number less than the range, may readily impede the formation of a higher-order, orderly aggregated structure and may impede the development of the shear-thickening properties. In contrast, the group as R¹, if containing carbon atoms in a number greater than the range, may invite higher interaction between or among hydrophobic chains to form an excessively rigid higher-order structure. This may cause insufficient recombination from a unit structure to a crosslinked structure even when strain is applied, where the recombination is associated with the deformation of the higher-order structure. The resulting liquid composition may less develop shear-thickening properties and, under certain circumstances, may crystallize and separate at low temperatures.

The liquid composition according to the present invention may contain the component (A) in a content of typically from about 1 to about 70 percent by weight, preferably from 2 to 50 percent by weight, particularly preferably from 3 to 30 percent by weight, and most preferably from 5 to 15 percent by weight. The liquid composition, if containing the component (A) in a content less than the range, may readily fail to sufficiently thicken or the liquid composition itself may readily separate to become heterogenous even upon the application of strain. In contrast, the liquid composition, if containing the component (A) in a content greater than the range, may have a remarkably high viscosity even without the application of strain. This may readily impair the practical value of the thickening effect of the liquid composition upon the application of strain, or may impede the formation of the higher-order structure itself, where the higher-order structure acts as a precondition for the function development.

The N-methyl-N-(2,3-dihydroxypropyl) fatty amide represented by Formula (1) can be produced by any of known methods such as following methods.

1) A method in which a fatty acid ester and an alkanolamine are allowed to react with each other in the presence of a basic catalyst (see U.S. Pat. No. 284,609).

2) A method in which a fatty acid is allowed to react with a large excess of an alkanolamine in the absence of a solvent (YUKAGAKU (Journal of Japan Oil Chemist's Society), vol. 24, No. 12, pp. 869-873, 1975).

3) A method in which a fatty acid and an alkanolamine are allowed to react with each other to give an amide ester, and a mixture of an alkanolamine and a basic catalyst dissolved or dispersed in the alkanolamine is added to the reaction system (JP-A No. S53-44513).

4) A method in which a fatty acid is added intermittently or continuously to an alkanolamine (JP-A No. H08-301827).

5) A method in which a fatty acid halide and an alkaline aqueous solution are added dropwise to an alkanolamine with cooling and allowing them to react with each other (Schotten-Baumann reaction).

Among them, the method 1) is preferred herein. In particular, the method 1) is preferably performed in a manner as follows. Specifically, an amine represented by Formula (3) is allowed to react with a fatty acid ester represented by Formula (4b) in an amount of 1.2 moles or less per mole of the amine in the presence of a basic catalyst. This gives an N-methyl-N-(2,3-dihydroxypropyl) fatty amide represented by Formula (1). Formulae (3), (4b), and (1) are expressed as follows:

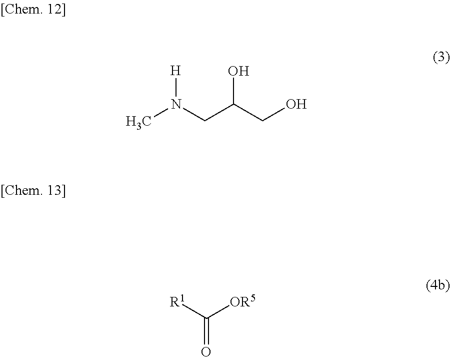

where R¹ represents C₅-C₂₀ straight or branched chain alkyl or alkenyl; and R⁵ represents a hydrocarbon group,

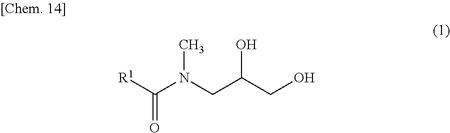

where R¹ is as defined above. The method is preferred because of enabling selective production of the target compound from highly safe starting materials under mild reaction conditions.

R¹ in Formula (4b) corresponds to R¹ in Formula (1).

In Formula (4b), R⁵ represents a hydrocarbon group and is exemplified by C₁-C₂₀ straight chain, branched chain, or cyclic saturated or unsaturated alkyl such as methyl, ethyl, propyl, allyl, isopropyl, butyl, amyl, hexyl, cyclohexyl, octyl, ethylhexyl, decyl, dodecyl, cetyl, stearyl, and icosyl. Among them, C₁ or C₂ alkyl is preferred herein. This is because the alkyl gives a low-molecular-weight alcohol as a by-product in the reaction, and such low-molecular-weight alcohol can be removed under mild conditions and less gives concerns of bad odor. In addition, the alkyl is easily available.

Specifically, the fatty acid ester represented by Formula (4b) is exemplified by methyl laurate, ethyl laurate, and 2-ethylhexyl laurate.

The fatty acid ester represented by Formula (4b) may be used in an amount of typically about 1.2 moles or less (e.g., from 0.95 to 1.20 moles), preferably from 0.96 to 1.05 moles, and particularly preferably from 0.96 moles to less than 1.03 moles per mole of the amine represented by Formula (3). The fatty acid ester represented by Formula (4b), if used in an amount greater than the range, may cause a side reaction to proceed and cause the N-methyl-N-(2,3-dihydroxypropyl) fatty amide represented by Formula (1) to be produced in a lower yield.

The basic catalyst is exemplified by sodium hydroxide, potassium hydroxide, sodium methoxide and a methanol solution thereof, potassium t-butoxide, and lithium hydroxide. The basic catalyst may be used in an amount of typically from about 0.001 mole to about 0.1 mole and preferably from 0.003 to 0.05 mole, per mole of the amine represented by Formula (3).

The reaction may be performed at a temperature of typically from about 60° C. to about 150° C., preferably from 70° C. to 130° C., and particularly preferably from 80° C. to 100° C. The reaction may be performed for a time typically from about 1 to about 60 hours and preferably from 10 to 50 hours. The reaction is preferably performed under reduced pressure typically at about 0.1 to about 600 mmHg and preferably at 1.0 to 400 mmHg. The reaction may be performed in an any atmosphere that does not adversely affect the reaction. In an embodiment, the reaction is performed at a low degree of pressure reduction in its early stages. In this embodiment, the reaction is preferably performed after the step of replacing the inside atmosphere of a reactor with an inert gas atmosphere such as nitrogen or argon atmosphere, where the reactor has been charged with starting materials. This is preferred for better hue and better catalytic activity.

After the completion of the reaction, a reaction product may be separated/purified by a separation means such as filtration, concentration, distillation, extraction, crystallization, recrystallization, or column chromatography, or a separation means as any combination of them. For example, the reaction product may be separated/purified by allowing a solid adsorbent to adsorb a catalyst-derived metal component and the amine represented by Formula (3) each remained in a crude reaction mixture and filtering the resulting reaction mixture, followed by recrystallization. The solid adsorbent is preferably one that can adsorb both the metal and the amine and can be separated by filtration or another means after the adsorption operation. The solid adsorbent for use herein can be suitably selected from a variety of ion exchange resins and powdery or particulate inorganic solid adsorbents.

Component (B)

The component (B) for use herein is the N-methyl-N-(2-hydroxypropyl) fatty amide ester represented by Formula (2).

In Formula (2), R² and R³ independently represent, identically or differently, C₅-C₂₀ straight or branched chain alkyl or alkenyl and are exemplified as with R¹.

In particular, R² and R³ in Formula (2) are each preferably C₉-C₁₅ straight chain alkyl and are more preferably identical groups (groups of the same kind). This is preferred from the viewpoints of fluidity and temperature stability. The groups as R² and R³, if containing carbon atoms in a number less than the range, may impede the formation of the higher-order structure and may readily cause the liquid composition to less develop shear-thickening properties. In contrast, the groups as R² and R³, if containing carbon atoms in a number greater than the range, may invite higher interaction between or among the hydrophobic chains to form an excessively rigid higher-order structure. This may cause insufficient recombination from a unit structure to a crosslinked structure even upon the application of strain, where the recombination is associated with the deformation of the higher-order structure. The resulting liquid composition may less develop shear-thickening properties and, under certain circumstances, may crystallize and separate at low temperatures.

The liquid composition according to the present invention may contain the component (B) in a content of typically from about 0.01 to about 10 percent by weight, preferably from 0.02 to 5 percent by weight, and particularly preferably from 0.03 to 3 percent by weight. The liquid composition, if containing the component (B) in a content less than the range, may tend to less thicken or gelate even upon the application of strain. In contrast, the composition, if containing the component (B) in a content greater than the range, may have remarkably high initial viscosity and elastic modulus to become a gel-like material. This may cause the composition to deteriorate in properties as a practical shear-thickening material.

The ratio (weight ratio) of the component (A) to the component (B) in the liquid composition is typically from about 99:1 to about 70:30, preferably from 99:1 to 80:20, more preferably from 98:2 to 80:20, particularly preferably from 97:3 to 90:10, and most preferably from 96:4 to 91:9.

The N-methyl-N-(2-hydroxypropyl) fatty amide ester represented by Formula (2) may be produced typically by a method as follows. In this method, an amine represented by Formula (3) is allowed to react with a fatty acid ester represented by Formula (4a) in an amount of 1.95 moles or more (e.g., 1.95 to 2.95 moles, preferably from 1.97 to 2.10 moles) per mole of the amine. The reaction is performed in the presence of a basic catalyst. This gives an N-methyl-N-(2-hydroxypropyl) fatty amide ester represented by Formula (2a). Formulae (3), (4a), and (2a) are expressed as follows:

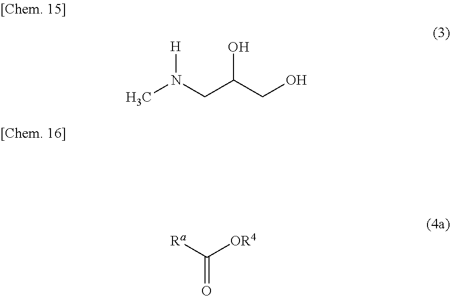

where R^{a} represents C₅-C₂₀ straight or branched chain alkyl or alkenyl; and R⁴ represents a hydrocarbon group,

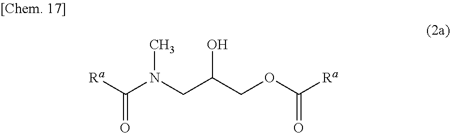

where R^{a} is as defined above.

In Formula (4a), R^{a} represents C₅-C₂₀ straight or branched chain alkyl or alkenyl and is exemplified as with R¹ in Formula (1).

In Formula (4a), R⁴ represents a hydrocarbon group and is exemplified as with R⁵ in Formula (4b). Specifically, the fatty acid ester represented by Formula (4a) is exemplified by methyl laurate, ethyl laurate, and 2-ethylhexyl laurate.

The basic catalyst is exemplified as above. The basic catalyst may be used in an amount of typically from about 0.001 mole to about 0.1 mole and preferably from 0.005 to 0.1 mole, per mole of the amine represented by Formula (3).

The reaction may be performed at a temperature of typically from 90° C. to 150° C., more preferably from 100° C. to 140° C., and particularly preferably from 110° C. to 130° C. The reaction may be performed for a time not critical. However, the reaction, if to be performed for an excessively short time, may require a large amount of the basic catalyst. In contrast, the reaction, if to be performed for an excessively long time, may readily cause the product to have inferior hue. To prevent these, the reaction may be performed for a time of typically from about 10 to about 100 hours and preferably from 20 to 70 hours. The reaction is preferably performed under reduced pressure typically at about 0.1 to about 600 mmHg and preferably at 1.0 to 400 mmHg. The reaction may be performed in any atmosphere that does not adversely affect the reaction. In an embodiment, the reaction is performed at a low degree of pressure reduction in its early stages. In this embodiment, the reaction is preferably performed after an upstream step of replacing the inside atmosphere of a reactor with an inert gas atmosphere such as nitrogen or argon atmosphere, where the reactor has been charged with starting materials. This is preferred for better hue and better catalytic activity.

After the completion of the reaction, a reaction product may be separated/purified in a manner as above. Specifically, the reaction product may be separated/purified by a separation means such as filtration, concentration, distillation, extraction, crystallization, recrystallization, or column chromatography, or a separation means as any combination of them.

The N-methyl-N-(2,3-dihydroxypropyl) fatty amide represented by Formula (1) and the N-methyl-N-(2-hydroxypropyl) fatty amide ester represented by Formula (2) may be produced together by a method as follows. Specifically, an amine represented by Formula (3) is allowed to react with a fatty acid ester represented by Formula (4a) in an amount of from 0.95 mole to less than 1.95 moles per mole of the amine, where the reaction is performed in the presence of a basic catalyst. This gives both an N-methyl-N-(2,3-dihydroxypropyl) fatty amide represented by Formula (1a) and an N-methyl-N-(2-hydroxypropyl) fatty amide ester represented by Formula (2a). Formulae (3), (4a), (1a), and (2a) are expressed as follows:

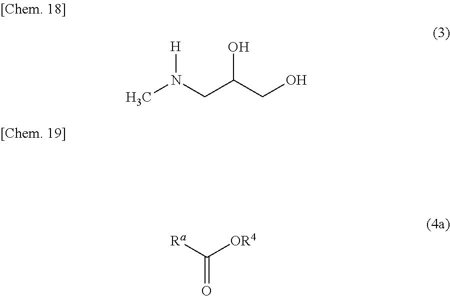

where R^{a} represents C₅-C₂₀ straight or branched chain alkyl or alkenyl; and R⁴ represents a hydrocarbon group,

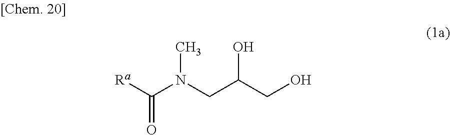

where R^{a} is as defined above,

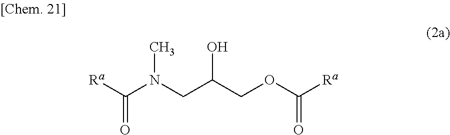

where R^{a} is as defined above. This method can give the fatty amide and the fatty amide ester together in weight fractions suitable for a liquid composition having shear-thickening properties.

The fatty acid ester represented by Formula (4a) may be used in an amount of typically from about 0.95 mole to less than about 1.95 moles, preferably from 1.0 to 1.5 moles, and particularly preferably from 1.03 to 1.2 moles, per mole of the amine represented by Formula (3).

The basic catalyst is exemplified as above. The basic catalyst may be used in an amount of typically from about 0.001 to about 0.1 mole and preferably from 0.003 to 0.05 mole, per mole of the amine represented by Formula (3).

The reaction may be performed at a temperature of typically from 90° C. to 150° C., more preferably from 100° C. to 140° C., and particularly preferably from 110° C. to 130° C. The reaction may be performed for a time not critical. However, the reaction, if performed for an excessively short time, may require a large amount of the basic catalyst; and, in contrast, the reaction, if performed for an excessively long time, may readily cause the product to have inferior hue. To prevent these, the reaction may be performed for a time of typically from about 1 to about 60 hours and preferably from 3 to 40 hours. The reaction is preferably performed under reduced pressure typically at about 0.1 to about 600 mmHg and preferably at 1.0 to 400 mmHg. The reaction may be performed in any atmosphere that does not adversely affect the reaction. In an embodiment, the reaction is performed at a low degree of pressure reduction for a considerably long time in early stages thereof so as to prevent bumping of the reaction mixture. In this embodiment, the reaction is preferably performed after starting materials are charged in a reactor and the inside atmosphere of the reactor is replaced with an inert gas atmosphere such as nitrogen or argon atmosphere. This is preferred for better hue and better catalytic activity.

After the completion of the reaction, a reaction product may be separated/purified in a manner as above by a separation means such as filtration, concentration, distillation, extraction, crystallization, recrystallization, or column chromatography, or by a separation means as any combination of them.

The method gives a mixture including the N-methyl-N-(2,3-dihydroxypropyl) fatty amide represented by Formula (1) and the N-methyl-N-(2-hydroxypropyl) fatty amide ester represented by Formula (2), in which the ratio (weight ratio) of the former to the latter is typically from about 99:1 to about 70:30, preferably from 99:1 to 80:20, more preferably from 98:2 to 80:20, particularly preferably from 97:3 to 90:10, and most preferably from 96:4 to 91:9.

The N-methyl-N-(2,3-dihydroxypropyl) fatty amide represented by Formula (1) and the N-methyl-N-(2-hydroxypropyl) fatty amide ester represented by Formula (2) may be prepared separately using independent production facilities in independent production processes and then mixed. However, the N-methyl-N-(2-hydroxypropyl) fatty amide ester represented by Formula (2) is an amphiphilic compound containing two hydrophobic groups and one hydrophilic group per molecule and has a strong tendency to self-assembly. For example, the compound itself may form a gel. This may cause the compound to offer inferior handleability upon production. To prevent this, the method capable of producing the N-methyl-N-(2,3-dihydroxypropyl) fatty amide represented by Formula (1) and the N-methyl-N-(2-hydroxypropyl) fatty amide ester represented by Formula (2) together is preferably employed. This is because the method can be performed by a simple production process to efficiently give the target compounds.

Component (C)

The component (C) for use herein is an oily substance and may be selected from known or common oily substances. The component (C) is exemplified by hydrocarbon oils such as liquid paraffin, squalane, n-octane, n-heptane, cyclohexane, and isododecane; ether oils such as dioctyl ether, ethylene glycol monolauryl ether, ethylene glycol dioctyl ether, and glycerol monooleyl ether; ester oils such as octyldodecyl myristate, isopropyl palmitate, butyl stearate, myristyl myristate, isopropyl myristate, di-2-ethylhexyl adipate, diisopropyl sebacate, neopentyl glycol dicaprate, and tricaproin; saturated branched chain higher (C₁₀-C₂₂) alcohols such as isostearyl alcohol and octyldodecanol; higher (C₁₀-C₂₂) fatty amides such as lauryl-lauramide and butyl-lauramide; vegetable oils such as olive oil, soybean oil, and cottonseed oil; silicone oils such as dimethylpolysiloxanes, cyclic dimethylpolysiloxanes, methylphenylpolysiloxanes, amino-modified silicones, epoxy-modified silicones, carboxy-modified silicones, alcohol-modified silicones, alkyl-modified silicones, polyether-modified silicones, and fluorine-modified silicones; fluorinated oils such as perfluoroalkylethyl phosphates, perfluoroalkyl polyoxyethylene phosphates, perfluoropolyethers, and polytetrafluoroethylenes. The liquid composition may contain each of them alone or in combination.

Among them, the liquid composition preferably contains any of hydrocarbon oils, of which straight or branched chain hydrocarbon oils containing carbon atoms in a number similar to those of R¹ to R³ in the compound represented by Formula (1) or Formula (2). Specifically, straight or branched chain hydrocarbon oils containing carbon atoms in a number of from 5 to 20, and preferably from 9 to 15 are preferred. This is preferred because the resulting liquid composition can satisfactorily form a precursory higher-order structure having high fluidity under no strain, but can readily undergo a change of the structure to a crosslinked higher-order structure and can rapidly thicken or gelate upon the application of strain.

The liquid composition according to the present invention may contain the component (C) in a content of typically from about 1 to about 50 percent by weight, preferably from 2 to 30 percent by weight, and particularly preferably from 3 to 20 percent by weight. The liquid composition, if containing the component (C) in a content out of the range, may often become nonuniform and may readily separate.

Component (D)

The component (D) for use herein is water. The water may be either of hard water and soft water and may be selected typically from pure water, industrial water, service water (tap water), ion-exchanged water, and distilled water.

The liquid composition according to the present invention may contain the component (D) in a content of typically from about 50 to about 98 percent by weight, preferably from 70 to 97 percent by weight, and particularly preferably from 75 to 95 percent by weight. The liquid composition, if containing the component (D) in a content less than the range, may less behave as a non-Newtonian fluid (i.e., more behave as a Newtonian fluid) and may tend to less thicken or gelate even upon the application of strain. In contrast, the liquid composition, if containing the component (D) in a content greater than the range, may readily fail to maintain its uniformity to separate, or may tend to less thicken or gelate even upon the application of strain.

In addition to the components (A) to (D), the liquid composition according to the present invention may further contain an additional component (E) within a range not adversely affecting the development of the rheology control function according to the present invention. The additional component (E) is exemplified by a variety of amphiphilic substances, oil-soluble additives, solvents (e.g., alcohols and polyhydric alcohols), antioxidants, humectants (moisturizers), pigment powders, flavors, dyes, organic or inorganic ultraviolet absorbers, antiseptic agents (preservatives), skin-whitening agents, and plant extracts. The liquid composition may contain each of them alone or in combination as the component (E).

The amphiphilic substances are exemplified by anionic surfactants, cationic surfactants, imidazoline amphoteric surfactants, amphoteric surfactants, and amino acid surfactants. The anionic surfactants are exemplified by alkyl sulfate salts such as sodium lauryl sulfate and potassium lauryl sulfate; alkyl ether sulfate salts such as triethanolamine polyoxyethylene lauryl sulfate; N-acylsarcosine salts such as sodium N-lauroylsarcosinate; higher fatty amide sulfonate salts such as sodium N-myristoyl-N-methyltaurate and sodium N-stearoyl-N-methyltaurate; phosphate salts such as sodium monostearyl phosphate, sodium polyoxyethylene oleyl ether phosphates, and sodium polyoxyethylene stearyl ether phosphates; sulfosuccinate salts such as sodium di-2-ethylhexylsulfosuccinate; alkylbenzenesulfonate salts such as linear sodium dodecylbenzenesulfonate and linear triethanolamine dodecylbenzenesulfonate; and N-acylglutamate salts such as monosodium N-lauroylglutamate, monosodium N-stearoylglutamate, disodium N-stearoylglutamate, and monosodium N-myristoyl-L-glutamate. The cationic surfactants are exemplified by alkyltrimethylammonium salts such as stearyltrimethylammonium chloride and lauryltrimethylammonium chloride; dialkyldimethylammonium salts such as dioctadecyldimethylammonium chloride; trialkylmethylammonium salts; and alkylamine salts. The imidazoline amphoteric surfactants are exemplified by sodium 2-undecyl-N,N-(hydroxyethylcarboxymethyl)-2-imidazoline and disodium 2-cocoyl-2-imidazolinium hydroxide-1-carboxyethyloxy. The amphoteric surfactants are exemplified by 2-heptadecyl-N-carboxymethyl-N-hydroxyethylimidazolinium betaine, lauryl dimethylaminoacetic acid betaine, alkyl-betaines, amidobetaine, and sulfobetaine. The amino acid surfactants are exemplified by N-laurylglycine and N-lauryl-β-alanine.

The oil-soluble additives are exemplified by sphingosines such as sphingosine, dihydrosphingosine, phytosphingosine, dehydrosphingosine, dehydrophytosphingosine, sphingadienine, and N-methyl-derivatives or N,N-dimethyl-derivatives of them; sterols such as cholesterol, cholesterol sulfate, polyoxyethylene cholesterol, stigmasterol, and ergosterol; and 1-(2-hydroxyethylamino)-3-isostearyloxy-2-propanol.

The alcohols and polyhydric alcohols are exemplified by ethanol, isopropanol, ethylene glycol, 1,2-propanediol, propylene glycol, 1,3-butanediol, 1,4-butanediol, 1,2-pentanediol, glycerol, trimethylolpropane, and pentaerythritol.

The antioxidants are exemplified by hindered phenolic antioxidants and phosphorus antioxidants. The hindered phenolic antioxidants are exemplified by products available under the trade names of HP-136, Irganox 1010, Irganox 1076, Irganox 1330, Irganox 3114, and Irganox 3125 (each from Ciba Specialty Chemicals Corporation, trademarks); ADK STAB AO-20, ADK STAB AO-50, ADK STAB AO-60, ADK STAB AO-80, ADK STAB AO-30, and ADK STAB AO-40 (each from ADEKA CORPORATION, trademarks), BHT (from Takeda Pharmaceutical Co., Ltd., trademark); Cyanox 1790 (from Cyanamid Company, trademark); and Sumilizer GP, Sumilizer GM, Sumilizer GS, and Sumilizer GA-80 (each from Sumitomo Chemical Co., Ltd., trademarks). The phosphorus antioxidants are exemplified by products available under the trade names of IRAGAFOS 168, IRAGAFOS 12, IRAGAFOS 38, IRAGAFOS P-EPQ, and IRAGAFOS 126 (each from Ciba Specialty Chemicals Corporation, trademarks); ADKSTAB 329K, ADKSTAB PEP-36, ADKSTAB PEP-8, ADKSTAB HP-10, ADKSTAB 2112, ADKSTAB 260, and ADKSTAB 522A (each from ADEKA CORPORATION, trademarks); Weston 618, Weston 619G, and Weston 624 (each from General Electric Company (GE), trademarks).

In addition, the liquid composition according to the present invention may further contain any of water-soluble or oil-soluble polymeric compounds depending on the intended use. The water-soluble polymeric compounds are exemplified by poly(vinyl alcohol)s, polyacrylamides, poly(vinyl methyl ether)s, polyisopropylacrylamides, polyethylene oxides, cellulose derivatives (e.g., methylcellulose, ethylcellulose, methyl hydroxypropyl cellulose, hydroxypropyl cellulose, and hydroxyethyl cellulose), starch-based modified polymers, propylene glycol alginate, guar gum, locust bean gum, tragacanth gum, gelatin, casein, dextrin, xanthan gum, pullulan, and starch. The oil-soluble polymeric compounds are exemplified by cyclopentasiloxane, dimethicone, alkyldimethicones, high-polymer methylpolysiloxanes, polybutenes, hydrogenated polyisobutenes, and poly(butyl acrylate)s. The liquid composition may contain the water-soluble or oil-soluble polymeric compound(s) in an amount of 10 percent by weight or less and preferably 5 percent by weight or less, based on the total amount of the liquid composition. When the liquid composition contains two or more different water-soluble or oil-soluble polymeric compounds, the term “amount” refers to the total amount thereof. The liquid composition, if containing the water-soluble or oil-soluble polymeric compound in an amount greater than the range, may place a larger burden on the environment to cause environmental pollution.

The liquid composition according to the present invention may be prepared by blending the components (A) to (D) and, as needed and optionally, the additional component (E), heating them as needed (e.g., at about 40° C. to about 80° C.), and stirring and mixing them using a general-purpose agitator (mixer) to give a uniform composition, or by the use typically of an ultrasonic emulsification device or a high-pressure emulsification device. The agitator is exemplified by single-screw or multi-screw extruders, kneaders, dissolvers, homogenizers, and rotor mixers.

The liquid composition according to the present invention has such shear-thickening properties as to thicken or gelate upon the application of strain. Assume that the liquid composition has a first storage modulus (storage elastic modulus) [G′ (Pa)] at a shear rate of 350 rad/s and a second storage modulus [G′ (Pa)] at a shear rate of 1 rad/s at a temperature of from 10° C. to 50° C. In this case, the liquid composition may have a difference between the first and second storage moduli of typically 20 Pa or more, preferably 30 or more, and particularly preferably 40 or more. Further assume that the liquid composition has a first loss modulus (loss elastic modulus) [G″ (Pa)] at a shear rate of 350 rad/s and a second loss modulus [G″ (Pa)] at a shear rate of 1 rad/s at a temperature of from 10° C. to 50° C. In this case, the liquid composition may have a difference between the first and second loss moduli of typically 10 Pa or more, preferably 15 Pa or more, and particularly preferably 20 Pa or more.

The liquid composition according to the present invention has reversible rheological properties and, even when once thickened or gelated, can have a low viscosity and offer high fluidity as recovered. The fluidity recovery can be performed typically by heating up to about 50° C. and cooling in a stationary state, or by leaving the composition stand at room temperature for about one month. In addition, the resulting liquid composition offering recovered fluidity can thicken or gelate again with good sensitivity upon another application of strain.

The liquid composition according to the present invention, as having the shear-thickening properties, does not scatter even when being traveled at a high speed on a production line so as to provide better productivity. In addition, the liquid composition enables precise printing even when subjected to high-speed printing. In an embodiment, the liquid composition is used typically as a cosmetic base material. In this embodiment, the liquid composition behaves as a high-fluidity liquid typically in a container, but can thicken or gelate upon the application of strain immediately before use. The strain application may be performed typically by shaking by hand or by discharging the composition from a container having a nozzle mechanism, such as a pump foamer (foaming pump container). The liquid composition can provide, to a consumer, a product that can be used in a non-conventional manner. Specifically, the product offers excellent usability without running down when taken in hand or applied to the skin and has a practical value. In addition, the product is allowed to have such an entertainment factor that the user oneself can change the state of the product before use. Accordingly, the liquid composition according to the present invention may be suitably usable, as a rheology control material, typically in or for impact absorbing material fillers, soling materials, viscoelastic abrasive media, materials for devices with silencing function (noise-reducing function), filling materials for display deformation restraining layers, printer inks, coating compositions, cosmetic base materials, and dental materials.

### EXAMPLES

The present invention will be illustrated in further detail with reference to several examples below. It should be noted, however, that the examples are by no means intended to limit the scope of the present invention.

### Example 1

### Production of 3-lauroylmethylamido-1,2-propanediol

There was prepared a 1-L four-neck flask equipped with a reflux condenser, a thermometer housed in a sheath tube, and a three-way stopcock for switching between a vent line and a vacuum pump decompression line. The reflux condenser included a nitrogen inlet line with a three-way stopcock for switching. In the flask, 300.10 g (1.40 mol) of methyl laurate and 151.62 g (1.44 mol) of 3-(N-methylamino)-1,2-propanediol (supplied by Daicel Corporation) were placed, and the reactor (flask) was placed under a nitrogen purge while raising the inside temperature to 70° C. and stirring. The mixture was combined with 6.30 g (0.028 mol) of a 28% sodium methoxide solution in methanol, the inside temperature was raised to 90° C., and the pressure was gradually reduced to 500 mmHg while monitoring the distilling state so as to prevent methanol bumping. While maintaining the temperature at 90° C., the reactor was then gradually decompressed to 100 mmHg, and at a lapse of 40 hours, it was verified by GC analysis that the amount of residual methyl laurate was 0.1% or less, followed by cooling the mixture down to 70° C.

The mixture was combined with 80.48 g of an inorganic synthetic adsorbent (trade name Kyowaad 700, supplied by Kyowa Chemical Industry Co., Ltd.), stirred for 5 hours, and filtered through a celite/filter paper using a hot-water-jacketed vacuum filter. The filtrate was combined with ethanol washings, concentrated and dried under reduced pressure on a rotary evaporator, and yielded 380.93 g of a wax-like pale-yellowish white product.

The prepared product was recrystallized from acetone and yielded 297.13 g of 3-lauroylmethylamido-1,2-propanediol as a needle-crystal white powder (Product 1) in a yield of 78.3%. The product was subjected to ¹H-NMR and IR analyses and identified to have the target molecular structure.

### Example 2

### Production of 1-(3-lauroylmethylamido-2-hydroxypropyl)laurate

There was prepared a 1-L four-neck flask equipped with a reflux condenser, a thermometer housed in a sheath tube, and a three-way stopcock for switching between a vent line and a vacuum pump decompression line. The reflux condenser included a nitrogen inlet line with a three-way stopcock for switching. In the flask, 342.97 g (1.60 mol) of methyl laurate and 85.38 g (0.81 mol) of 3-(N-methylamino)-1,2-propanediol (supplied by Daicel Corporation) were placed, and the reactor (flask) was placed under a nitrogen purge while raising the inside temperature to 70° C. and stirring. The mixture was combined with 1.55 g (0.008 mol) of a 28% sodium methoxide solution in methanol. The inside temperature was raised to 110° C., and the pressure was gradually reduced to 600 mmHg while monitoring the distilling state so as to prevent methanol bumping. While maintaining the temperature at 110° C., the pressure was gradually reduced to 150 mmHg, and at a lapse of 65 hours, it was verified by GC analysis that the amount of residual methyl laurate was 0.1% or less, followed by cooling the mixture down to 80° C. The mixture was combined with 380 g of ethanol, stirred until the mixture became uniform, and placed in a refrigerator at −10° C. to precipitate as crystals. The crystals were filtered, washed, dried, and yielded 88.55 g of 1-(3-lauroylmethylamido-2-hydroxypropyl) laurate as a crystalline white powder (Product 2) in a yield of 23.6%. The prepared product was identified by ¹H-NMR to have the target molecular structure.

### Example 3

### Production of mixture of 3-lauroylmethylamido-1,2-propanediol and 1-(3-lauroylmethylamido-2-hydroxypropyl)laurate

There was prepared a 1-L four-neck flask equipped with a reflux condenser, a thermometer housed in a sheath tube, and a three-way stopcock for switching between a vent line and a vacuum pump decompression line. The reflux condenser included a nitrogen inlet line with a three-way stopcock for switching. In the flask, 331.17 g (1.55 mol) of methyl laurate and 157.71 g (1.50 mol) of 3-(N-methylamino)-1,2-propanediol (supplied by Daicel Corporation) were placed, and the reactor (flask) was placed under a nitrogen purge while raising the inside temperature to 70° C. and stirring. The mixture was combined with 3.377 g (0.015 mol) of a 28% sodium methoxide solution in methanol. The inside temperature was raised to 110° C., and the pressure was gradually reduced to 600 mmHg while monitoring the distilling state so as to prevent methanol bumping. While maintaining the temperature at 110° C., the pressure was gradually reduced to 150 mmHg. At a lapse of 28 hours, it was verified by GC analysis that the amount of residual methyl laurate was 0.1% or less, and the mixture was cooled down to 80° C.

The mixture was combined with 80.48 g of an inorganic synthetic adsorbent (trade name Kyowaad 700, supplied by Kyowa Chemical Industry Co., Ltd.), stirred for 5 hours, filtered through a celite/filter paper using a hot-water-jacketed vacuum filter, the filtrate was combined with ethanol washings, concentrated and dried under reduced pressure on a rotary evaporator, and yielded 408.85 g of a wax-like pale-yellowish white product (Product 3).

Product 3 was analyzed by ¹H-NMR and found to contain 3-lauroylmethylamido-1,2-propanediol and 1-(3-lauroylmethylamido-2-hydroxypropyl)]laurate in a weight ratio of 91.2:8.8.

### Example 4

### Production of mixture of 3-lauroylmethylamido-1,2-propanediol and 1-(3-lauroylmethylamido-2-hydroxypropyl)laurate

There was prepared a 1-L four-neck flask equipped with a reflux condenser, a thermometer housed in a sheath tube, and a three-way stopcock for switching between a vent line and a vacuum pump decompression line. The reflux condenser included a nitrogen inlet line with a three-way stopcock for switching. In the flask, 321.52 g (1.50 mol) of methyl laurate and 157.70 g (1.50 mol) of 3-(N-methylamino)-1,2-propanediol (supplied by Daicel Corporation) were placed, and the reactor (flask) was placed under a nitrogen purge while raising the inside temperature to 70° C. and stirring. The mixture was combined with 3.377 g (0.015 mol) of a 28% sodium methoxide solution in methanol, the inside temperature was raised to 110° C., and the pressure was gradually reduced to 600 mmHg while monitoring the distilling state so as to prevent methanol bumping. While maintaining the temperature at 110° C., the pressure was gradually reduced to 150 mmHg. At a lapse of 40 hours, it was verified by GC analysis that the amount of residual methyl laurate was 0.1% or less, and the mixture was cooled down to 80° C.

The mixture was combined with 43.2 g of an inorganic synthetic adsorbent (trade name Kyowaad 700, supplied by Kyowa Chemical Industry Co., Ltd.), stirred for 5 hours, filtered through a celite/filter paper using a hot-water-jacketed vacuum filter, the filtrate was combined with ethanol washings, concentrated and dried under reduced pressure on a rotary evaporator, and yielded 413.12 g of a wax-like pale-yellowish white product (Product 4).

Product 4 was analyzed by ¹H-NMR and found to contain 3-lauroylmethylamido-1,2-propanediol and 1-(3-lauroylmethylamido-2-hydroxypropyl) laurate in a weight ratio of 95.4:4.6.

### Comparative Example 1

### Synthesis of N-(2-hydroxy-3-hexadecyloxypropyl)-N-hydroxyethylhexadecanamide

A mixture of 8.325 g (300 mmol) of ethanolamine and 14.885 g of ethanol was heated to 70° C., combined with 7.443 g (24.6 mmol) of hexadecyl glycidyl ether added dropwise over one hour, further stirred at 70° C. for 3 hours, concentrated and dried on a rotary evaporator, reprecipitated from methanol, and yielded 5.615 g (14.44 mmol) of N-(2-hydroxy-3-hexadecyloxypropyl)ethanolamine in a yield of 62.8%. This was combined with 87 mg (0.77 mmol) of potassium t-butoxide, heated to 80° C., and yielded a liquid mixture. The mixture was combined with 1.392 g (5.15 mmol) of methyl palmitate and decompressed at a pressure of 300 mmHg to 20 mmHg for 2 hours to remove a low-boiling substance. This operation was repeated three times. The resulting crude reaction mixture was combined with n-heptane, from which solids were collected by filtration, and yielded 7.557 g of a crude product. Of the crude product, 7.000 g were subjected to purification with a silica gel chromatograph using a heptane-ethyl acetate solvent mixture and yielded 6.290 g (10.45 mmol) of N-(2-hydroxy-3-hexadecyloxypropyl)-N-hydroxyethylhexadecanamide (Product 5) in a yield of 73.0%. Product 5 was subjected to ¹H-NMR and ¹³C-NMR analyses using a solvent mixture of CDCl₃ and DMSO-d₆ to identify its structure and was found to be a mixture including two rotamers having different configuration with respect to substituents bonded to the central nitrogen element. This identification was performed in the solvent at room temperature.

### Comparative Example 2

### Production of 1-[3-(N-laurylcarbamoylmethylamino)-2-hydroxypropyl]laurate

An aliquot (2.835 g (26.97 mmol)) of 3-(N-methylamino)-1,2-propanediol (supplied by Daicel Corporation) was combined with 5.670 g of ethanol and cooled on an ice bath. This was combined with 5.533 g (26.18 mmol) of dodecyl isocyanate added dropwise over one hour, further stirred at room temperature for one hour, stirred with heating to reflux for one hour, combined with 2.649 g (26.18 mmol) of triethylamine, cooled on an ice bath, combined with 6.095 g (26.18 g) of lauroyl chloride added dropwise over one hour, further stirred for 2 hours with cooling on an ice bath, stirred at room temperature for 5 hours, decompressed to remove a low-boiling substance, dried, and yielded 11.15 g of a solid.

The total amount of the solid was subjected to purification with a silica gel chromatograph using a heptane-ethyl acetate solvent mixture and yielded 4.590 g (10.45 mmol) of a mixture (Product 6) of 1-[3-(N-laurylcarbamoylmethylamino)-2-hydroxypropyl]laurate and 2-[3-(N-laurylcarbamoylmethylamino)-1-hydroxypropyl]laurate in a yield of 36.5% as a regioisomer mixture developing one spot in TLC. The two components were identified as a mixture even after further recrystallization and TLC under different conditions. This indicates that the components are probably substances that promptly exchange structures with each other in a solution at room temperature and have such a property as to constitute an equilibrium mixture and to be hardly isolated from each other by a regular purification method.

### Comparative Example 3

### Production of N-methyl-N-(2-hydroxyethyl)lauroylamide

There was prepared a 1-L four-neck flask equipped with a reflux condenser, a thermometer housed in a sheath tube, and a three-way stopcock for switching between a vent line and a vacuum pump decompression line. The reflux condenser included a nitrogen inlet line with a three-way stopcock for switching. In the flask, 85.739 g (400 mmol) of methyl laurate and 30.945 g (0.412 mmol) of 2-(methylamino)ethanol were placed, and the reactor (flask) was placed under a nitrogen purge while raising the inside temperature to 70° C. and stirring. The mixture was combined with 0.772 g (4 mmol) of a 28% sodium methoxide solution in methanol, the inside temperature was raised to 90° C., and the pressure was gradually reduced to 500 mmHg while monitoring the distilling state so as to prevent methanol bumping. While maintaining the temperature at 90° C., the pressure was gradually reduced to 100 mmHg. At a lapse of 10 hours, it was verified by GC analysis that the amount of residual methyl laurate was 0.1% or less, and the mixture was cooled down to 70° C.

The mixture was combined with 20.6 g of an inorganic synthetic adsorbent (trade name Kyowaad 700, supplied by Kyowa Chemical Industry Co., Ltd.), stirred for 5 hours, filtered through a celite/filter paper using a hot-water-jacketed vacuum filter, the filtrate was combined with ethanol washings, concentrated and dried under reduced pressure on a rotary evaporator, and yielded 94.503 g of a wax-like pale-yellowish white product. This was recrystallized from acetone and yielded 68.436 g of N-methyl-N-(2-hydroxyethyl)lauroylamide (Product 7) as a needle crystal white powder in a yield of 66.5%. This was subjected to ¹H-NMR analysis and found to have the target molecular structure.

### Examples 5 to 11 and Comparative Examples 4 to 11

Components including any of the products prepared in Examples 1 to 4 and Comparative Examples 1 were mixed in predetermined amounts (in gram) given in Table 1 by an operation indicated as Process-1 and yielded compositions. The resulting compositions were subjected to Process-2 and Process-3 to examine their properties.

Process-1

According to a formulation given in Table 1, components excluding pure water-2 were weighed in a 10-mL graduated screw-cap test tube, stirred and dispersed on a vortex mixer, then stirred using a rotor mixer (trade name MIX ROTOR VMRC-5, supplied by AS ONE Corporation) at 45° C. for one hour until the mixture appeared to be visually uniform, combined with a predetermined amount of the pure water-2 added dropwise with stirring on a vortex mixer, and yielded a composition. The resulting composition was stored in a thermostat at 40° C. for 10 hours or longer, further stored in a thermostat at 25° C. for about 3 hours, and how the composition is was visually examined.

Process-2

Each of the screw-cap test tubes containing the compositions was shaken hard by hand at room temperature (20° C. to 30° C.) to apply strain to the composition, and immediately thereafter, how the composition is was visually examined.

Process-3

Each of the screw-cap test tubes containing the compositions was immersed in water in a constant-temperature water bath at 50° C. to be warmed for one hour, further stored in a thermostat at 25° C. for about 3 hours, and how the composition is was visually examined.

The results are together shown in the following tables. The isododecane in the tables was a product available under the trade name of MARUKASOL R (from Maruzen Petrochemical Co., Ltd.).

TABLE 1

| | | Example 5 | Example 6 | Example 7 | Example 8 | Example 9 | Example 10 | Example 11 |
|---|---|---|---|---|---|---|---|---|
| Liquid | Pure water-1 | 0.210 | 0.210 | 0.210 | 0.190 | 0.190 | 0.190 | 0.210 |
| composition | Isododecane | 0.350 | 0.400 | 0.450 | 0.800 | 1.000 | 0.400 | 0.400 |
| | Product 1 | 0.475 | 0.475 | 0.475 | — | — | 0.475 | 0.475 |
| | Product 2 | 0.025 | 0.025 | 0.025 | — | — | 0.015 | 0.015 |
| | Product 3 | — | — | — | 1.000 | — | — | — |
| | Product 4 | — | — | — | — | 1.000 | — | — |
| | Product 5 | — | — | — | — | — | 0.010 | 0.010 |
| | Product 6 | — | — | — | — | — | — | — |
| | Product 7 | — | — | — | — | — | — | — |
| | Pure water-2 | 7.790 | 7.790 | 7.790 | 7.010 | 7.010 | 7.010 | 7.790 |
| Evaluation | Process-1 | liquid | liquid | liquid | liquid | liquid | liquid | liquid |
| | Process-2 | gel | gel | gel | gel | gel | gel | gel |
| | Process-3 | liquid | liquid | liquid | liquid | liquid | liquid | liquid |

TABLE 2

| | | Com- | Com- | | | | | | |
|---|---|---|---|---|---|---|---|---|---|
| | | parative | parative | Comparative | Comparative | Comparative | Comparative | Comparative | Comparative |
| | | Example 4 | Example 5 | Example 6 | Example 7 | Example 8 | Example 9 | Example 10 | Example 11 |
| Liquid | Pure water-1 | 0.190 | 0.190 | 0.190 | 0.190 | 0.190 | 0.210 | 0.190 | 0.190 |
| composition | Isododecane | 0.800 | 1.000 | 0.400 | 0.480 | 0.560 | 0.400 | 0.800 | 0.800 |
| | Product 1 | 1.000 | 0.950 | 0.475 | 0.570 | 0.665 | 0.475 | — | — |
| | Product 2 | — | — | — | — | — | — | 0.050 | 0.050 |
| | Product 3 | — | — | — | — | — | — | — | — |
| | Product 4 | — | — | — | — | — | — | — | — |
| | Product 5 | — | 0.050 | 0.025 | 0.030 | 0.035 | — | — | — |
| | Product 6 | — | — | — | — | — | 0.025 | — | — |
| | Product 7 | — | — | — | — | — | — | 0.950 | — |
| | Pure water-2 | 7.010 | 7.010 | 7.790 | 7.010 | 7.010 | 7.790 | 7.010 | 7.010 |
| Evaluation | Process-1 | liquid | liquid | liquid | liquid | liquid | liquid | liquid | liquid |
| | | | | | | | | | (separated) |
| | Process-2 | liquid | liquid | liquid | liquid | liquid | liquid | liquid | liquid |
| | | | | | | | | | (separated) |
| | Process-3 | liquid | liquid | liquid | liquid | liquid | liquid | liquid | liquid |
| | | | | | | | | | (separated) |

The results demonstrated as follows. The compositions prepared in Examples 5 to 11 contained low-molecular organic compounds alone as organic compounds and included water as a component in a content of greater than 70% of the composition. Despite of this, the compositions were found to have shear-thickening properties. Specifically, the compositions, when placed in a container and shaken by hand at room temperature (20° C. to 30° C.) to be applied with strain, had an abruptly increased viscosity and gelated. In addition, the compositions prepared in Examples 5 to 7 were found to have a reversible behavior. Specifically, assume that the compositions were allowed to gelate once, and then heated to 50° C. and cooled, or left stand at room temperature for about one month. In this case, the compositions had a lower viscosity, offered recovered high fluidity, and behaved as liquids. The resulting liquids, when shaken by hand at room temperature, gelated again. The compositions prepared in the examples were found to have shear-thickening properties regardless of their production processes. Specifically, the compositions in Examples 5 to 7 were prepared by separately preparing the component (A) and the component (B) and mixing them with each other. The compositions in Examples 8 and 9 were prepared by synthesizing a mixture of the component (A) and the component (B) in one pot. The compositions in Examples 10 and 11 were prepared by further adding another amphiphilic substance in addition to the component (A) and the component (B).

In contrast, as shown in Table 2, each of the compositions prepared in Comparative Examples 4 to 11 did not have such shear-thickening properties as to gelate upon the application of strain.

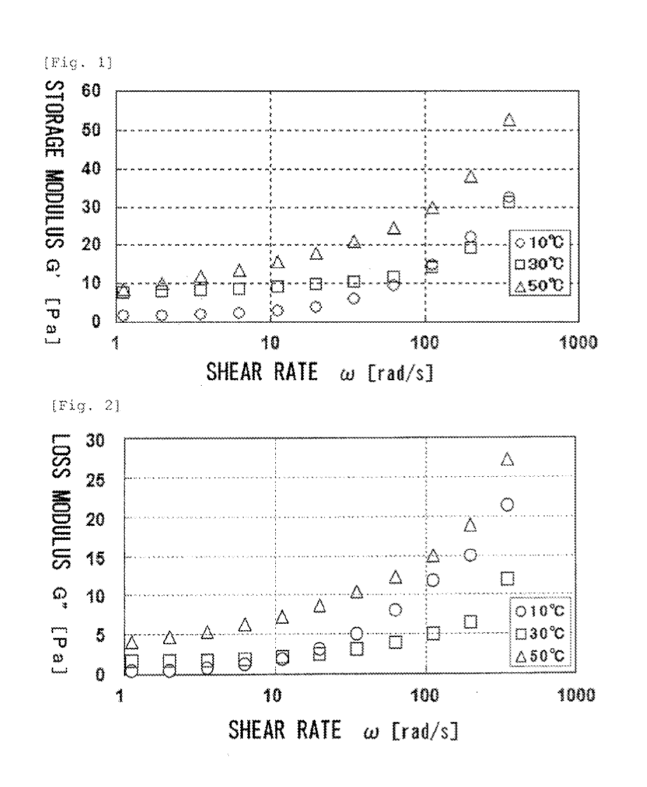

The composition prepared in Example 8 was further subjected to a dynamic viscoelasticity measurement at 10° C., 30° C., and 50° C. at shear rates (ω) in the range from 0.1 to 500 rad/s using an MCR301 Rheometer (supplied by Anton Paar GmbH) (see FIGS. 1 and 2).

The result revealed that, with an increasing shear rate, the composition tended to have both remarkably increased storage modulus (G′) and loss modulus (G″) and to gelate (namely to become resistant to deformation).

Accordingly, the liquid compositions according to the present invention have a rheology control function so as to offer various physical properties ranging from a liquid state to a gel state according to the use and can maintain the function even when combined with a variety of additives depending on the use. The compositions act as rheology control materials of wide application.

### INDUSTRIAL APPLICABILITY

The liquid compositions according to embodiments of the present invention have such properties as to develop shear-thickening properties with good sensitivity and can maintain the properties over a long period of time. The liquid compositions do not require the use of polymeric compounds placing large burden on the environment and are environmental-friendly. The liquid compositions according to the present invention are advantageously usable as rheology control materials in or for materials requiring shear-thickening properties. Such materials are exemplified by impact absorbing material fillers, soling materials, viscoelastic abrasive media, materials for devices with silencing function (noise-reducing function), filling materials for display deformation restraining layers, printer inks, coating compositions, cosmetic base materials, and dental materials.

1. A liquid composition comprising following components:
(A) an N-methyl-N-(2,3-dihydroxypropyl) fatty amide represented by Formula (1): 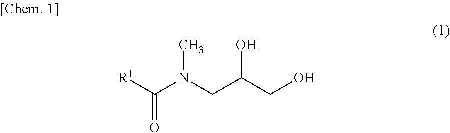
wherein R¹ represents C₅-C₂₀ straight or branched chain alkyl or alkenyl;
(B) an N-methyl-N-(2-hydroxypropyl) fatty amide ester represented by Formula (2): 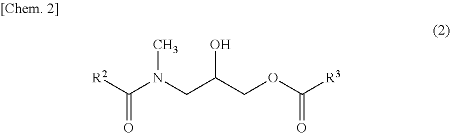
wherein R² and R³ independently represent, identically or differently, C₅-C₂₀ straight or branched chain alkyl or alkenyl;
(C) an oily substance; and
(D) water.

2. The liquid composition according to claim 1,
wherein a ratio (weight ratio) of the component (A) to the component (B) is from 99:1 to 70:30.

3. The liquid composition according to one of claims 1 and 2,
wherein the component (C) comprises a straight or branched chain hydrocarbon oil.

4. The liquid composition according to claim 1,
wherein the liquid composition has a first storage modulus [G′ (Pa)] at a shear rate of 350 rad/s, a second storage modulus [G′ (Pa)] at a shear rate of 1 rad/s, where a difference between the first storage modulus and the second storage modulus is 20 Pa or more,
wherein the liquid composition has a first loss modulus [G″ (Pa)] at a shear rate of 350 rad/s and a second loss modulus [G″ (Pa)] at a shear rate of 1 rad/s, where a difference between the first loss modulus and the second loss modulus is 10 Pa or more, and
wherein the storage moduli and loss moduli are determined at a temperature of from 10° C. to 50° C.

5. The liquid composition according to claim 1, further comprising a component (E),
the component (E) comprising an amphiphilic substance excluding compounds belonging to the components (A) and (B).

6. A method for producing a fatty amide and a fatty amide ester, the method comprising
allowing an amine represented by Formula (3) to react with a fatty acid ester represented by Formula (4a) in an amount of from 0.95 mole to less than 1.95 moles per mole of the amine in the presence of a basic catalyst to give both an N-methyl-N-(2,3-dihydroxypropyl) fatty amide represented by Formula (1a) and an N-methyl-N-(2-hydroxypropyl) fatty amide ester represented by Formula (2a),
Formulae (3), (4), (1a), and (2a) expressed as follows: 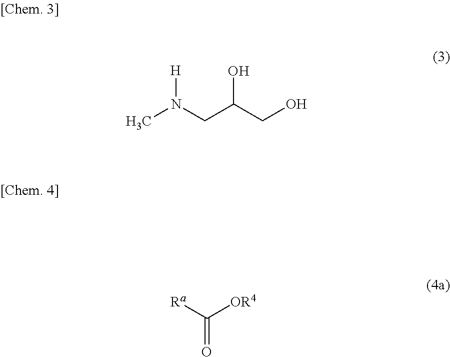
wherein R^{a} represents C₅-C₂₀ straight or branched chain alkyl or alkenyl; and R⁴ represents a hydrocarbon group, 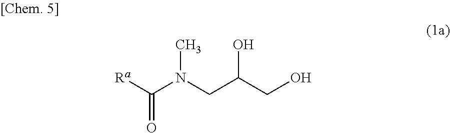
wherein R^{a} is as defined above, 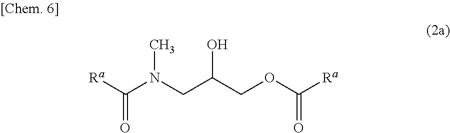
wherein R^{a} is as defined above.

7. A method for producing a fatty amide, the method comprising
allowing an amine represented by Formula (3) to react with a fatty acid ester represented by Formula (4b) in an amount of 1.2 moles or less per mole of the amine in the presence of a basic catalyst to give an N-methyl-N-(2,3-dihydroxypropyl) fatty amide represented by Formula (1),
Formulae (3), (4b), and (1) expressed as follows: 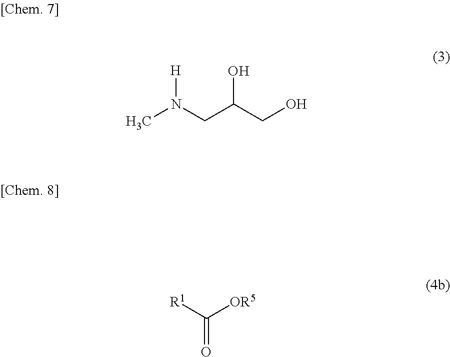
wherein R¹ represents C₅-C₂₀ straight or branched chain alkyl or alkenyl; and R⁵ represents a hydrocarbon group, 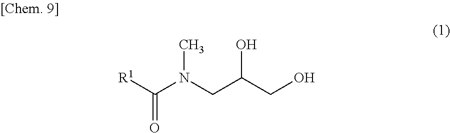
wherein R¹ is as defined above.

8. A method for producing a liquid composition, the method comprising:
preparing a mixture of an N-methyl-N-(2,3-dihydroxypropyl) fatty amide represented by Formula (1a) and an N-methyl-N-(2-hydroxypropyl) fatty amide ester represented by Formula (2a) by the method for producing a fatty amide and a fatty amide ester according to claim 6; and
mixing the mixture with an oily substance and water,
Formulae (1a) and (2a) expressed as follows: 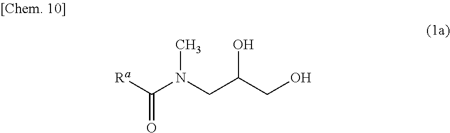
wherein R^{a} represents C₅-C₂₀ straight or branched chain alkyl or alkenyl, 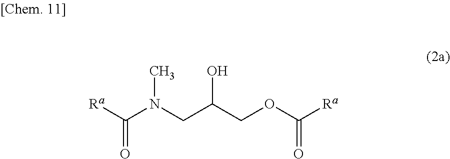
wherein R^{a} is as defined above.
